(19) United States
(12) Patent Application Publication
(10) Pub. No.: US 2014/0180256 A1
(43) Pub. Date: Jun. 26, 2014

(54) SINGLE COIL CONTACT GUIDEWIRE CONNECTOR

(71) Applicant: VOLCANO CORPORATION, San Diego, CA (US)

(72) Inventor: Bret Millett, Folsom, CA (US)

(73) Assignee: VOLCANO CORPORATION, San Diego, CA (US)

(21) Appl. No.: 14/132,413

(22) Filed: Dec. 18, 2013

Related U.S. Application Data
(60) Provisional application No. 61/745,271, filed on Dec. 21, 2012.

(51) Int. Cl. A61M 25/09

(52) U.S. Cl. CPC A61M 25/09
USPC 604/528

(57) ABSTRACT

The present invention generally relates to guidewire connectors. The invention can involve a housing that includes a channel. The invention can also involve at least one contact element configured to hold a guidewire inside the housing by pressing the guidewire against the channel.

### RELATED APPLICATION

This application claims the benefit of and priority to U.S. Provisional Ser. No. 61/745,271, filed Dec. 21, 2012, which is incorporated by reference in its entirety.

### FIELD OF THE INVENTION

The present invention generally relates to a guidewire connector for use in medical procedures.

### BACKGROUND

Cardiovascular disease frequently arises from the accumulation of atheromatous deposits on inner walls of vascular lumen, particularly the arterial lumen of the coronary and other vasculature, resulting in a condition known as artherosclerosis. These deposits can have widely varying properties, with some deposits being relatively soft and others being fibrous and/or calcified. In the latter case, the deposits are frequently referred to as plaque. These deposits can restrict blood flow, and in more severe cases, lead to myocardial infarction.

The assessment and treatment of cardiovascular disease often involves determining the difference in pressure across a coronary lesion to assess the extent that the lesion impedes the delivery of oxygen to the heart. A guidewire configured with a pressure sensor at the distal end is typically used to measure pressure across the lesion. The electrical signals from the pressure sensor are lead through conductors embedded in the guidewire to a connector at the proximal end of the guidewire. The connector is connected to an interface, which converts signals from the pressure sensor into a form readable by the operator.

Many conventional guidewire connectors retain the guidewire through the use of two or more coils that abut the guidewire. The guidewire is inserted into the connector and through the application of sufficient force, the guidewire overcomes the resistance of the multiple coils, pushing them sufficiently apart to allow a length of the guidewire to pass. The resistance of the coils then holds the guidewire in place. The guidewire, however, is fragile and often breaks as it is pushed against the coils. This can further complicate already difficult procedures by delaying or preventing diagnosis and treatment.

### SUMMARY

The invention provides guidewire connectors that offer decreased resistance to insertion of a guidewire into a vascular catheter. The connectors are configured to hold a guidewire inside a housing element by pressing the guidewire against a channel inside the housing. The connectors or contact elements switch between a deployed state in which they are pressed against the channel and an undeployed state in which they are held away from the channel walls.

Guidewire connectors of the invention offer little or no resistance during insertion of the guidewire. Accordingly, the connectors of the present invention are much more amenable for use with delicate guidewires. During insertion of the guidewire, the contact elements are in an undeployed state, allowing the guidewire to pass unhindered. Once the guidewire is positioned appropriately, the contact elements can then be deployed, holding the guidewire in place.

In certain aspects of the invention, the contact element is a coil which holds the guidewire against the channel when the contact element is in a deployed state. While connectors of the invention include at least one contact element or coil, it is also contemplated that certain embodiments of the invention utilize a plurality of contact elements to hold the guidewire in place. In certain aspects, the contact element includes a notch or a radial indentation. The radial indentation is configured to run parallel to an inserted guidewire. This indentation facilitates the retention of the guidewire without using excessive force by providing a groove in which the guidewire can rest as it is being held against the chamber. In further aspects of the invention, the indentation may also include a conductive surface in which a signal from the guidewire can be transmitted.

In certain aspects of the invention, the housing is not a monolithic unit but rather includes a distal portion and a proximal portion. The distal portion or the “nose” is the section into which the guidewire is inserted. The proximal end or the “body” is the section leading to an interface. The proximal end of the guidewire connector is closer to the operator. The distal region has an aperture into which the guidewire is inserted. The aperture is in communication with the channel so that a guidewire entering the aperture continues into the channel. In certain aspects of the invention, the aperture and channel together form a hyperbolic funnel. The hyperbolic funnel eliminates any oblique surfaces that may collide with the guidewire upon insertion, thereby reducing the incidence of breakage even further.

In certain aspects of the invention, the distal region and proximal region of the housing is configured to rotate independently of each other. In certain embodiments, the connector is configured such that rotation of either region can switch the contact elements from a deployed state to an undeployed state and vice versa. For example, by rotating the proximal region in one direction, the contact elements lock the inserted guidewire in place. By rotating the proximal region in an opposite direction, the contact elements release the guidewire.

In certain embodiments, the connector housing is further modified to enhance the overall usefulness of the device. In certain aspects, the housing is transparent or translucent, thereby allowing the operator to see the guidewire advance through the housing. In other aspects of the invention, the housing includes a guidewire stop inside the housing that ceases or impedes further advancement of the guidewire within the housing. This physical stop can provide tactile and visible feedback to the operator that the guidewire is fully inserted. In additional aspects of the invention, the channel within the housing contains bumps or protrusions that lightly hold a fully inserted guidewire when the contact elements are in the undeployed state.

### BRIEF DESCRIPTION OF THE DRAWINGS

FIGS. 1A and 1B illustrate an exemplary embodiment of the invention in an undeployed position and deployed position respectively, from a frontal perspective.

FIGS. 2A and 2B illustrate a conventional device of the prior art in an undeployed and deployed position respectively, from a side perspective.

FIGS. 3A, 3B, and 3C illustrate side, top, and frontal perspectives, respectively, of an exemplary embodiment of the invention in an undeployed and a deployed state.

FIG. 4 illustrates an exemplary contact element of the invention.

FIG. 5 depicts a single unit of multiple contact elements, according to certain embodiments.

FIG. 6 depicts a plurality of contact element units positioned on a lead frame and aligned with a contact strip, according to certain embodiments.

FIG. 7 exemplifies the consistent alignment and spacing between a plurality of contact elements positioned on a lead frame.

FIG. 8 illustrates a plurality of contact elements within a housing channel, according to certain embodiments.

FIG. 9 illustrates an exemplary guidewire connector.

FIGS. 10A and 10B illustrate the insertion of a guidewire into an exemplary embodiment of the invention.

FIGS. 11A and 11B illustrate a hyperbolic funnel in accordance with the invention and a non-hyperbolic funnel, respectively.

### DETAILED DESCRIPTION

The invention generally relates to a guidewire connector. More specifically, the invention relates to a guidewire connector that includes a housing comprising a channel and at least one contact element configured to hold a guidewire inside the housing by pressing the guidewire against the channel. As encompassed by the invention, the contact elements are able to switch between a deployed state and an undeployed state. In the deployed state, the contact elements are positioned such that they are able to retain the guidewire within the housing by pressing the guidewire against the channel. In the undeployed state, the contact elements withdraw from the channel, and therefore offer little or no resistance to the insertion or removal of the guidewire when in this state. This in contrast to many conventional guidewire connectors in which the guidewire is held between two or more coils that provide pressure against the sides of the guidewire. In these conventional devices, the coils actually resist the initial insertion of the guidewire. It is only when this initial resistance is surpassed through the use of greater force that the coils move apart, providing space to hold the guidewire. Unfortunately, the application of this greater force to overcome the resistance can easily break the fragile guidewire. This can complicate procedures and delay diagnosis and treatment.

Reference will now be made to the embodiments depicted in the provided Figures. It is to be understood that the invention is merely illustrated rather than limited by the provided Figures and that other modifications and embodiments are contemplated and encompassed.

The general operation of the provided devices is shown schematically in FIGS. 1A and 1B, which represent the device in an undeployed and deployed state, respectively. A guidewire 101 (shown from a frontal perspective) is inserted into the channel of the device housing (not shown in this figure for sake of simplicity). Below the guidewire 101 is a contact element 102 located within the channel. The contact element 102, here presented as a coil, is attached to a printed circuit board (PCB) 103, also located and fixed within the channel. The contact element 102 features a groove 104 configured to accommodate the guidewire 101. As shown in FIG. 1A, when the device is in the undeployed or disengaged state, the contact element 102 does not lie within the insertion/removal path of the guidewire. Therefore, the contact element 102 provides zero force resistance to the guidewire 101 during the insertion process.

In the deployed or engaged state, as shown in FIG. 1B, the PCB 103 is moved towards the guidewire 101, causing the contact element 102 to press against the guidewire 101 and hold the guidewire 101 against the channel wall. As shown in this embodiment, the guidewire 101, though pressed against the channel wall, is also held snugly within the groove 104 of the contact element 102. This ensures that the use of excessive force to retain the guidewire 101 is avoided. Without the groove 104, the focused localized force may cause the fragile guidewire to snap 101. Nonetheless, it is contemplated that in certain embodiments, the contact elements do not include a groove.

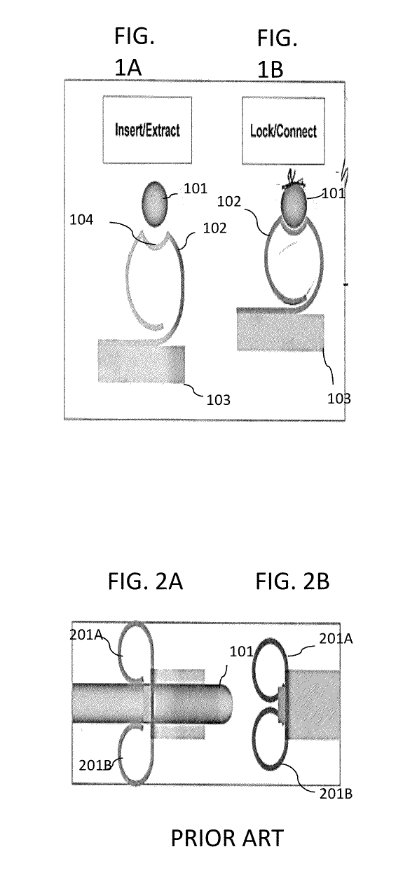

The operation of the provided device as shown in FIGS. 1A and 1B facilitates retention of the guidewire without breakage. This in contrast to many conventional guidewire connectors in which the guidewire is held between two or more coils 201A and 201B, such as those shown in FIGS. 2A and 2B, that provide pressure against the sides of the guidewire 101. In these conventional devices, the coils 201A and 201B actually resist the initial insertion of the guidewire 101. It is only when this initial resistance is surpassed through the use of greater force that the coils 201A and 201B move apart, providing space to hold the guidewire 101. Unfortunately, the application of this greater force to overcome the resistance can easily break the fragile guidewire.

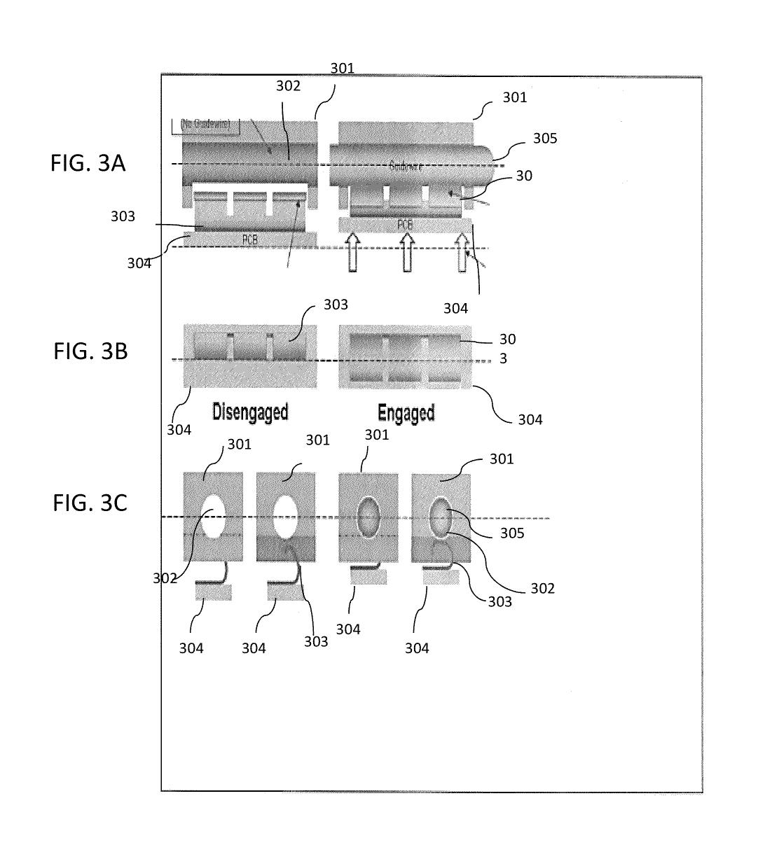

Further detail regarding the operation of the device is shown in FIGS. 3A, 3B, and 3C, which depict side, top, and frontal perspectives, respectively of the provided device in an undeployed (left-hand drawings) and deployed (right-hand drawings) state. The drawings to the left illustrate an exemplary device of the invention in a disengaged state, without an inserted guidewire. As shown, the provided device encompasses a housing 301 with a channel 302 located therein. Located within the channel 302 is a plurality of contact elements 303 connected to a PCB board 304. The contact elements 303 are configured to be “zero insertion force” style contacts when disengaged, meaning they offer no resistance to the insertion or removal of a guidewire. When a guidewire 305 has been inserted into the channel 302, the guidewire can be retained in the housing 301 by moving the PCB 304 towards the guidewire 305. This engages the coils of the contact element 303 to press the guidewire 305 against the wall of the channel 302, thereby retaining guidewire 305 within the housing 301. As particularly shown in FIGS. 3B and 3C, engaging the contact elements 303 by moving the PCB 304 causes the coils of the contact elements 303 to coil more, providing increased surface area and force against the guidewire 305.

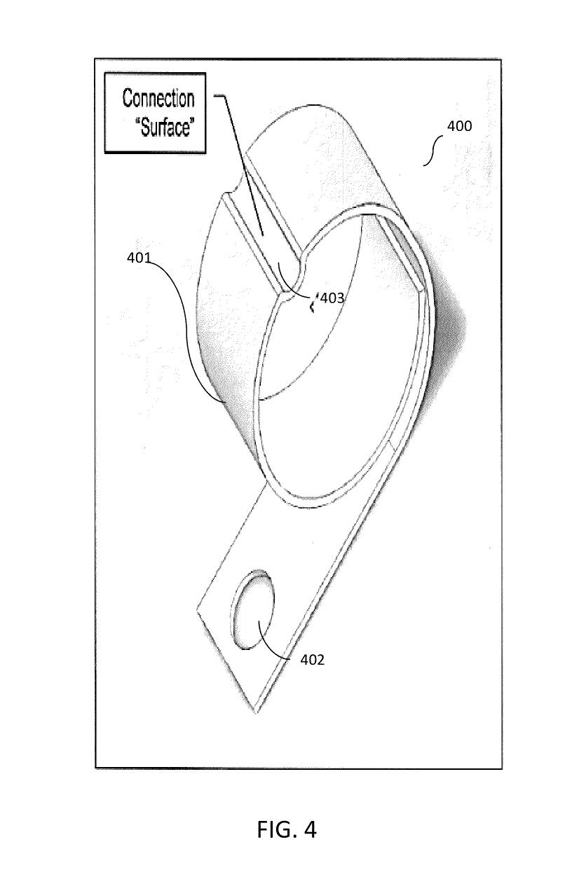

A exemplary contact element in accordance with the invention is provided in FIG. 4. As shown, the contact element 400 comprises a coil 401 that presses a guidewire against a channel wall in the provided device. The coil configuration allows the smooth application of pressure to the guidewire rather than a sharp application of force. In the embodiment shown, the contact element 400 comprises a hole 402 to facilitate screwing or soldering the contact element 400 to a surface, such as a PCB. Other means may also be used to affix the contact element to a surface. The exemplary contact element in FIG. 4 also features a notch or groove 403 configured to accommodate the guidewire and eliminate sharp surfaces in the contact element that may result in breaking the guidewire, rather than retaining it.

In certain embodiments, the contact element groove comprises a conductive surface that runs parallel to the guidewire. The conductive surface is comprised of a radial indentation along the contact surface that is parallel to the guidewire and features a radius matched to the guidewire. In certain embodiments, the major coil of the contact element is formed into a spring/cylinder that terminates with an offset, flat pedestal/Single Mount Type (SMT) contact surface.

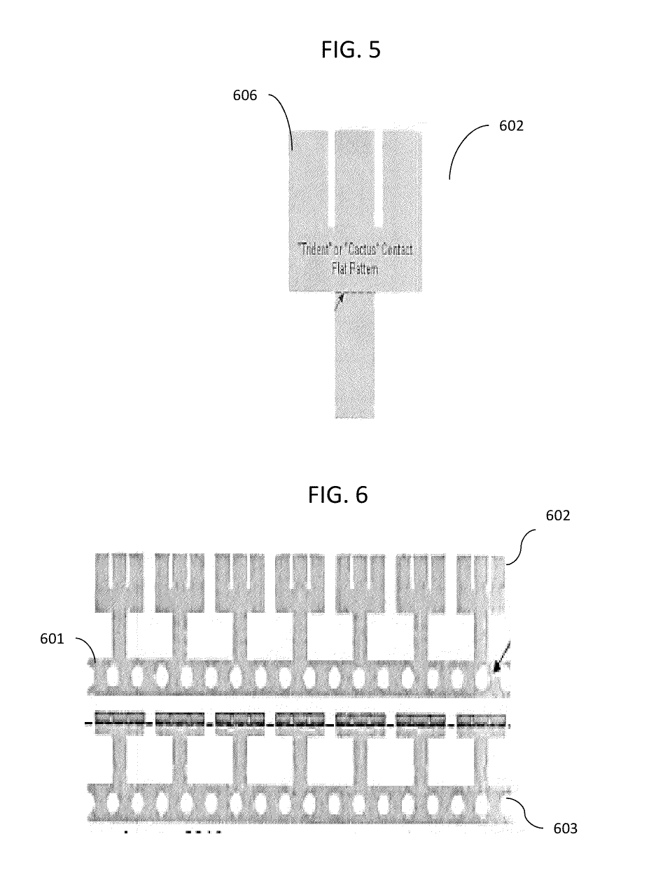

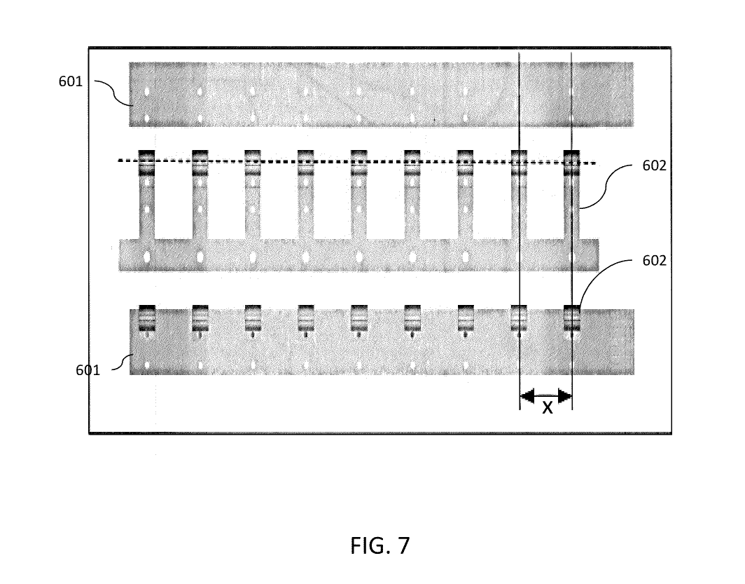

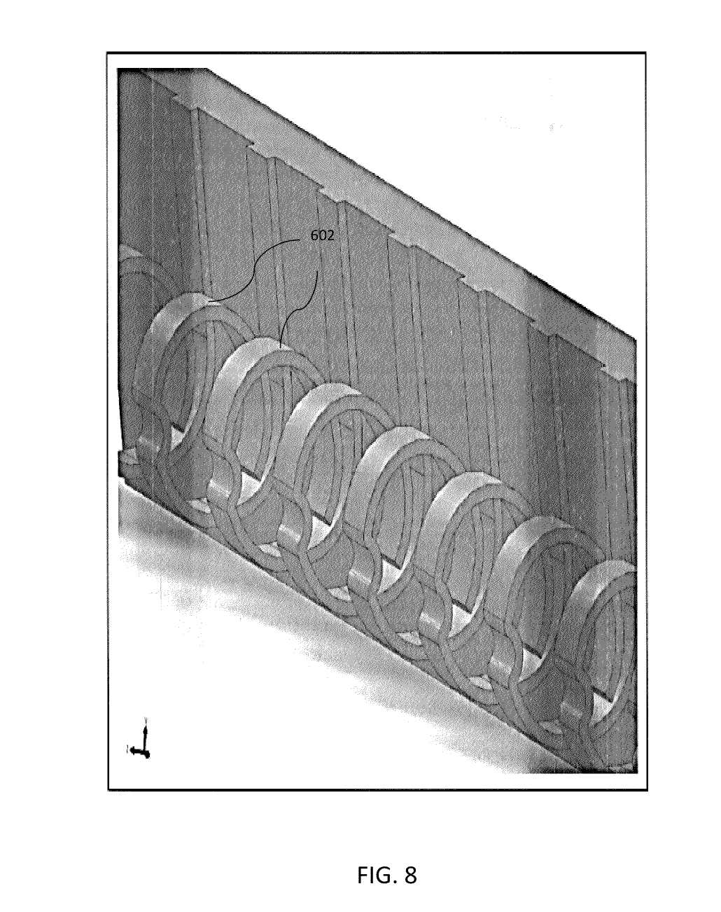

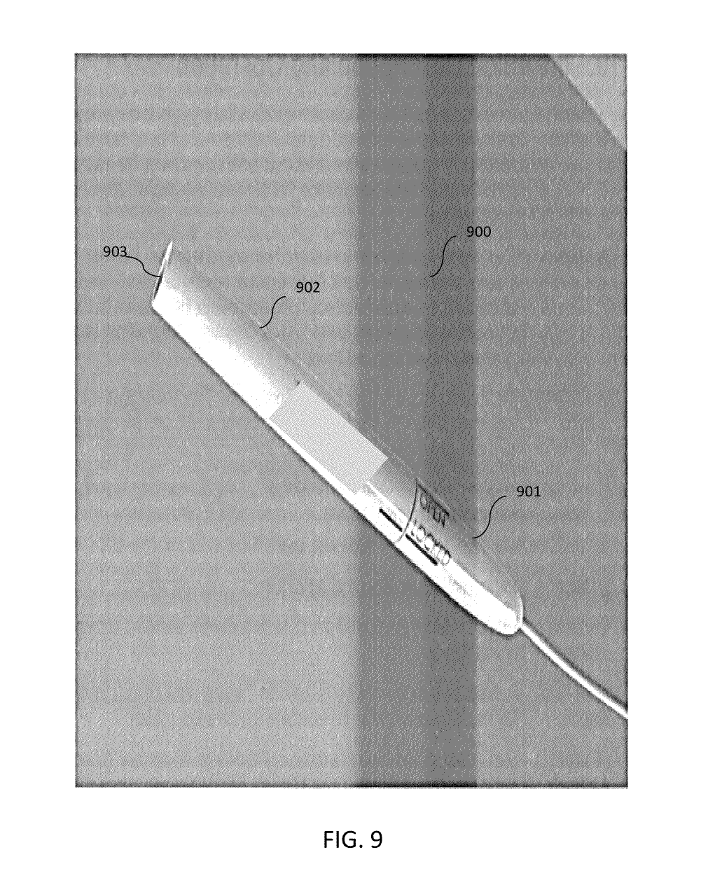

In certain embodiments, each contact is formed on a continuous lead-frame with fixed spacing between each contact element and fixed axial alignment between each contact element, as shown in FIG. 6. In certain embodiments, each contact element 602 attached to the lead frame 601 comprises a trident configuration of several individual coils 606, as shown in FIG. 5. A lead-frame 601 with multiple contact elements 602 is precision aligned to features on a contact PCB 603. The contact elements 602 from the lead-frame 601 are then gang-soldered onto the PCB 603 and subsequently separated from the lead-frame 601. This method of assembly can be used to generate a highly-precise array of contact elements as shown in FIG. 8, for implementation into the provided devices. The highly repeatable stamping and forming process just described produces uniform rows of contact elements 602 with fixed distance x between elements attached to a common lead-frame 601 prior to attachment to the PCB, as shown in FIG. 7.

Reference will now be made to the housing component of the provided guidewire connector. The housing can be prepared from any material known in the art, but in certain embodiments, the housing is prepared from a plastic or plastic polymer. In further aspects of the invention, the housing is prepared from a transparent or translucent plastic. This allows the operator to see the guidewire as it is inserted into the device. An exemplary embodiment of the housing is depicted in FIG. 9. As shown, the profile of the housing 900 is smooth and devoid of protrusions. In addition, the connector housing of certain embodiments is not a monolithic unit but rather comprises a proximal region 901 and a distal region 902. The distal component 902 or the “nose” is the section into which the guidewire is inserted. The proximal end 901 or the “body” leads to an interface which interprets signals received from the guidewire. The distal region 902 contains an aperture 903 into which the guidewire is inserted. In certain aspects, the distal component 902 is larger than the proximal component 901. For example, the nose length can be approximately 68 mm. The body length may be approximately 39 mm. The diameter at the nose tip may be approximately 9.6 mm. The diameter at which the nose and body meet may be 11.6 mm.

In certain aspects of the invention, the distal region and proximal region of the housing can rotate independently of each other. For example, one could hold the body in one hand and turn the nose with the other or hold the nose component and turn the body. In certain embodiments, the rotation of one region or the other can switch the contact elements between a deployed state and an undeployed state. For example, the housing component may be locked (engaged) or unlocked (disengaged) with one or two hands. In certain aspects, the transition from the locked position to the unlocked position may involve a rotation of 90 degrees. The amount of rotation can be adjusted as desired. In the lock position, the contact element detents will snap into place. In certain embodiments, the device is configured such that the locking of holding components in place elicits tactile and audible feedback. In the lock position, an inserted guidewire fits into the axially aligned indentation along the row of contact elements, shown for example in FIG. 8. In certain embodiments, clockwise rotation of one of the two housing components will lock the receptacle, while counter-clockwise rotation will unlock the receptacle, allowing the guidewire to be easily removed. As contemplated by the invention, the PCB and soldered contact elements press against the guidewire via operator rotation of one of the housing components, for example, the nose. The guidewire is thus pinned against the channel wall by the rigid proximity of the PCB and the spring force of the contacts.

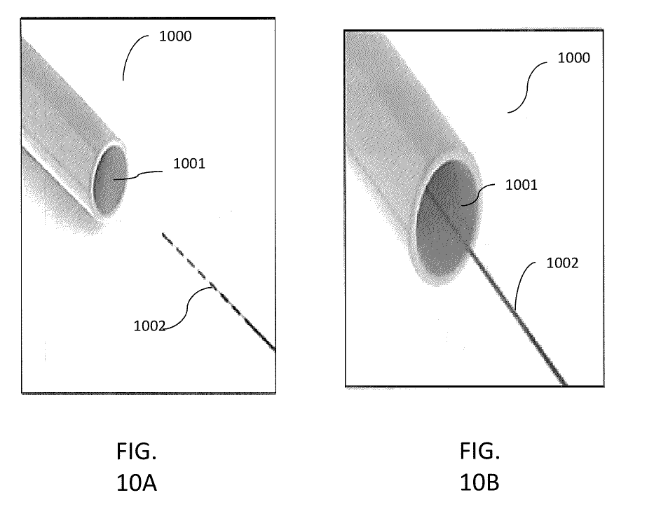

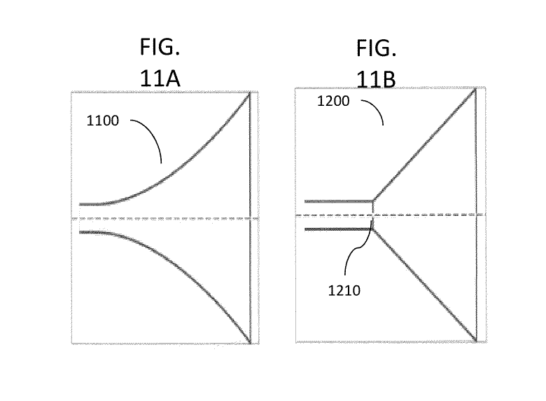

As shown in FIG. 10A, the provided connectors 1000 may feature a relative wide aperture 1001 relative to the size of the guidewire 1002. This facilitates the insertion of the guidewire 1002 into the device 1000, shown in FIG. 10B. The aperture 1001 is connected to the housing channel, so that a guidewire 1002 entering the aperture 1001 continues into the channel. In certain aspects of the invention, the aperature and channel are configured such that they together form a hyperbolic funnel, as shown in FIG. 11A. The hyperbolic funnel 1100 facilitates the smooth entrance of the guidewire into the channel. For comparative purposes, a non-hyperbolic funnel 1200 is shown in FIG. 11B. Although the opening of the non-hyperbolic funnel 1200 may be just as wide as the hyperbolic funnel 1100, the non-hyperbolic funnel 1200 contains a relatively sharp juncture 1210 between the funnel and the channel, which may break the delicate guidewire should the guidewire be inserted at an incorrect angle.

In certain embodiments, the connector housing is further modified to enhance the overall usefulness of the device. For example, the housing may include a guidewire stop inside the housing that ceases or impedes further advancement of the guidewire within the housing. This physical stop can provide tactile and visible confirmation to the operator that the guidewire has been fully inserted. In additional aspects of the invention, the channel within the housing contains bumps or protrusions that lightly hold a fully inserted guidewire when the contact elements are in the undeployed state. For example, bumps implemented in the guidewire channel between the two most proximal contacts would lightly hold a fully inserted guidewire when the device is in the unlocked position.

While particular embodiments of the present invention have been shown and described, modifications may be made, and it is therefore intended to cover in the appended claims, all such changes and modifications which fall within the true spirit and scope of the invention as defined by those claims.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A guidewire connector, the guidewire connector comprising:
a housing comprising a channel; and
at least one contact element configured to hold a guidewire inside the housing by pressing the guidewire against the channel.

2. The guidewire connector of claim 1, wherein the contact element is configured to have a deployed state and an undeployed state.

3. The guidewire connector of claim 2, wherein the deployed state comprises the contact element entering the channel and the undeployed state comprises the absence of the contact element in the channel.

4. The guidewire connector of claim 1, wherein the contact element is a coil.

5. The guidewire connector of claim 1, wherein the contact element comprises a radial indentation configured to run parallel to an inserted guidewire.

6. The guidewire connector of claim 5, wherein the radial indentation comprises a conductive surface.

7. The guidewire connector of claim 1, wherein the housing comprises a proximal region and a distal region.

8. The guidewire connector of claim 7, wherein the distal region contains an aperture in communication with the housing channel configured for the insertion of a guidewire.

9. The guidewire connector of claim 8, wherein the channel and aperture together comprise a hyperbolic funnel.

10. The guidewire connector of claim 7, wherein the distal region and proximal region can rotate independently of each other.

11. The guidewire connector of claim 10, wherein the rotation of either the proximal or distal region is configured to shift the contact element between the deployed state and undeployed state.

12. The guidewire connector of claim 7, wherein the distal region of housing is larger than the proximal region.

13. The guidewire connector of claim 1, wherein the housing is transparent or translucent.

14. The guidewire connector of claim 1, wherein the housing further comprises a stop configured to provide feedback to an operator upon full insertion of the guidewire.

15. The guidewire connector of claim 2, wherein the channel further comprises a plurality of protrusions configured to partially hold an inserted guidewire in place when the contact elements are in an undeployed state.
